(19) United States
(12) Patent Application Publication
(10) Pub. No.: US 2010/0286653 A1
(43) Pub. Date: Nov. 11, 2010

(54) REMOTE CONTROL DEVICE FOR USE WITH INSULIN INFUSION SYSTEMS

(75) Inventors: James Kubel, Sacramento, CA (US); Phillip Brooks, Morgan Hill, CA (US); Michael A. Cozzolino, Sacramento, CA (US)

Correspondence Address: DERGOSITS & NOAH LLP, Three Embarcadero Center, Suite 410, SAN FRANCISCO, CA, 94111, US

(73) Assignee: Axsol Incorporated, DBA Acess Solutions, Sacramento, CA (US)

(21) Appl. No.: 12/774,164

(22) Filed: May 5, 2010

Related U.S. Application Data
(60) Provisional application No. 61/213,129, filed on May 8, 2009.

(51) Int. Cl. A61M 37/00; G09B 23/28

(52) U.S. Cl. 604/504; 434/262; 604/500

(57) ABSTRACT

Methods and apparatuses providing accessibility options for the blind and poorly sighted for use with insulin therapy systems. A remote control device, with speech output capability and comprehensive speech menu system, may monitor or intercept data generated by a blood glucose meter, and delay, modify and retransmit said data to an insulin pump. The remote control device may also be used to program and set operating parameters of an insulin pump and also record data received from an insulin pump. The remote control device may also be used in conjunction with a personal computing device.

### CROSS-REFERENCE TO RELATED APPLICATIONS

This application claims priority to U.S. Provisional Patent Application No. 61/213,129, A Remote Control Device For Use With Insulin Infusion Systems, filed May 8, 2009, the entire contents of which are incorporated by reference.

### FIELD OF THE INVENTION

This invention relates to external infusion devices and systems that comprise an insulin pump and/or a blood glucose meter that utilize RF telemetry for inter-device communication and operation.

### BACKGROUND OF THE INVENTION

People with type 1 and some type 2 diabetes benefit immensely from the use of wearable external insulin pumps, and more so when used in conjunction with blood glucose meters. Insulin pumps more closely emulate the natural insulin generation of healthy individuals, than say those individuals that have diabetes and take insulin by injection or by pill form. Insulin pumps typically are small devices, about the size of a pager and are typically worn on a belt, but may be placed in a pocket or other area within other clothing areas so as to be not visible to the general public. Insulin pumps have a very small display, and 4 or 5 small buttons that allow a user to control the operation of the pump.

Proper management of diabetes requires constant vigil on the part of the user. A user may need to measure blood glucose levels as many as 10 or more times per day. If necessary the user will also enter information or commands into the insulin pump to control the flow of insulin to be commensurate with a given blood glucose reading. Patients with diabetes who have learned to manage their disease in conjunction with blood glucose meters and insulin pumps can live near normal lives, but must be constantly vigilant to maintain a healthy life.

Even with good management of diabetes, many individuals are still affected negatively by the disease. Diabetes can result in circulatory problems, amputation, kidney failure and blindness. There are some 25,000 people that go blind every year because of diabetes. What is needed is an improved system for allowing blind people to learn how to use a blood glucose meter and properly control an insulin pump with a remote control unit.

There are many problems with existing insulin pumps. One negative aspect of controlling typical insulin pumps is the requirement to access the front panel of the device. If worn in a concealed fashion this makes access to the controls more difficult, requiring removal of the pump from one's belt, pocket or an area of clothing. There are also problems with existing insulin pumps used with blood glucose meters. The meter transmits the blood glucose reading to an insulin pump, but there is no option to stop or pause that operation in the event of an error in the blood glucose reading by the meter.

Another negative aspect of current insulin pump therapy is that the devices cannot be safely operated by the blind or by people with very poor vision. The insulin pumps and remote control units have small screens and lack speech feedback. Thus, many people who use insulin pumps and who go blind cannot operate their insulin pumps and must rely on others for help. Depending on one's insurance or financial ability, assistance in managing their diabetes may simply not be available. Many newly blind diabetics find themselves facing a rapid deterioration of their health as related to their diabetic condition.

Another problem is communications incompatibility between blood glucose meters and insulin pumps made by various manufacturers. Some manufacturers have produced “talking” blood glucose meters that allow the blind and poorly sighted user to measure their blood glucose levels, but there are none yet that will easily transmit the blood glucose reading information to the insulin pumps. Yet another negative aspect of the current state of the art is that insulin pump users who go blind find that their physicians often take away their insulin pumps, for fear that they will make an insulin dosing mistake with the current poor state of blind accessibility of existing insulin therapy systems.

What is needed is a system that can provide insulin pump access to blind and vision impaired individuals and additionally be capable of teaching that individual to safely use the insulin pump to improve upon or eliminate the aforementioned negative aspects of current insulin therapy systems.

### SUMMARY OF THE INVENTION

The present invention is directed towards an insulin pump system that can include a remote control, a blood glucose meter, an insulin pump and a continuous blood glucose monitoring system. The system can be controlled by the remote control that emits audio instructions for training a user to use the remote control and use aids that help a user use the insulin pump and blood glucose meter. The remote control can communicate with the blood glucose meter and the insulin pump by RF communication. The remote control allows the user to operate a blood glucose meter and the insulin pump by providing speech guided menus that can be operated by a blind or visually impaired user. The remote control can have a size and shape that is a comfortably held and operated device, and includes control buttons and a speaker for speech, or earphone jack for private sound communication with the user. The buttons are preferably distinct in shape, position, raised markings or size so that a user can identify them by touch with the fingers. The remote control device also includes a processor connected to an RF transceiver. The remote control may also include a display capable of showing large easily read text and messages. The processor can also include software and firmware that allows the remote control to run operational programs for controlling the system.

When the remote control is used to transmit control and communications signals between the blood glucose meter and the insulin pump, it may need to be able to receive communication signals from the blood glucose meter and respond to these signals with an acknowledgment signal that is identical to that of the insulin pump. The remote control may also be able to provide a means to manually enter a blood glucose value and transmit this value to a compatible insulin pump, thereby emulating the function of a compatible blood glucose meter. The remote control can also transmit a variety of commands and operating parameters to a compatible insulin pump. For example, the remote control can receive usage data and operating parameter data from a compatible insulin pump. The remote control can provide a means of entering and transmitting user determined operating parameters and settings to a compatible insulin pump.

In an embodiment, the remote control device can be connected to a personal computer through a USB port, and through the use of appropriate software on the computer, can be made to perform all existing functions of the insulin infusion system. There are various additional aspects to the proposed invention. It should therefore be understood that the preceding is merely a brief summary of some possible embodiments and aspects of the proposed invention. Additional embodiments and aspects are referenced below. It should be further understood that numerous changes to the disclosed embodiments can be made without departing from the spirit or scope of the proposed invention.

### BRIEF DESCRIPTION OF THE DRAWINGS

FIG. 1 is a diagram of the remote control, insulin pump and blood glucose meter;

FIG. 2 is a block diagram of the remote control;

FIGS. 3a-3f illustrate the display of the remote control during menu scrolling;

FIGS. 4a-4d illustrate the display of the remote control during submenu displays;

FIG. 5 is a flow chart of the training process provided by the remote control;

FIG. 6 is a graphical illustration of a basal profile;

FIG. 7 is a graphical illustration of a standard bolus;

FIG. 8 is a graphical illustration of an extended bolus;

FIG. 9 is a graphical illustration of a combination bolus; and

FIG. 10 is a graphical illustration of a super bolus.

### DETAILED DESCRIPTION

In the following description, reference is made to the accompanying drawings which form a part hereof and which illustrate several embodiments of the invention. It is understood that other embodiments may be used and structural and operational changes may be made without departing from the scope of the invention.

### A. Remote Control Insulin Pump

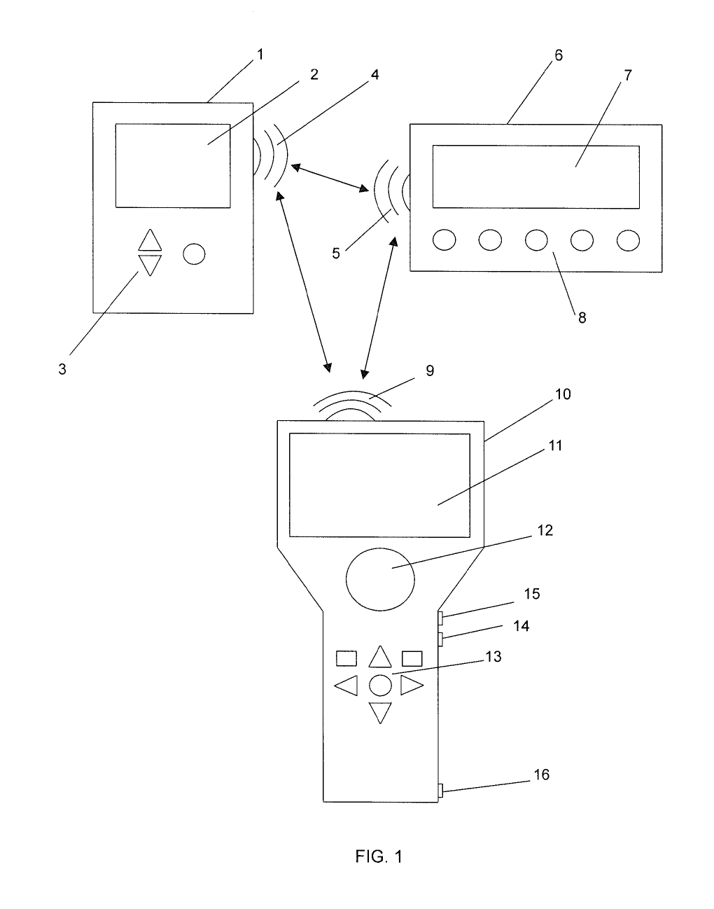

With reference to FIG. 1, the remote control 10 can transmit and receive compatible RF data from a blood glucose meter 1 and/or a compatible insulin pump 6. The remote control 10 can include: an RF transceiver 21, a display 11, a speaker 12, buttons 13, a headphone jack 14, USB jack 15 and an external power jack 16. The blood glucose meter 1 can include: a display 2, buttons 3 and an RF transceiver 4. In some embodiments, blood glucose information is transmitted via RF from the blood glucose meter 1 to the insulin pump 6 and the remote control 10. In an embodiment, the remote control 10 will speak and/or display the received blood glucose value. In other embodiments, the communications can be configured to transmit blood glucose information from the blood glucose meter 1 to the remote control 10 where it can be saved/examined and/or retransmitted to the insulin pump 6.

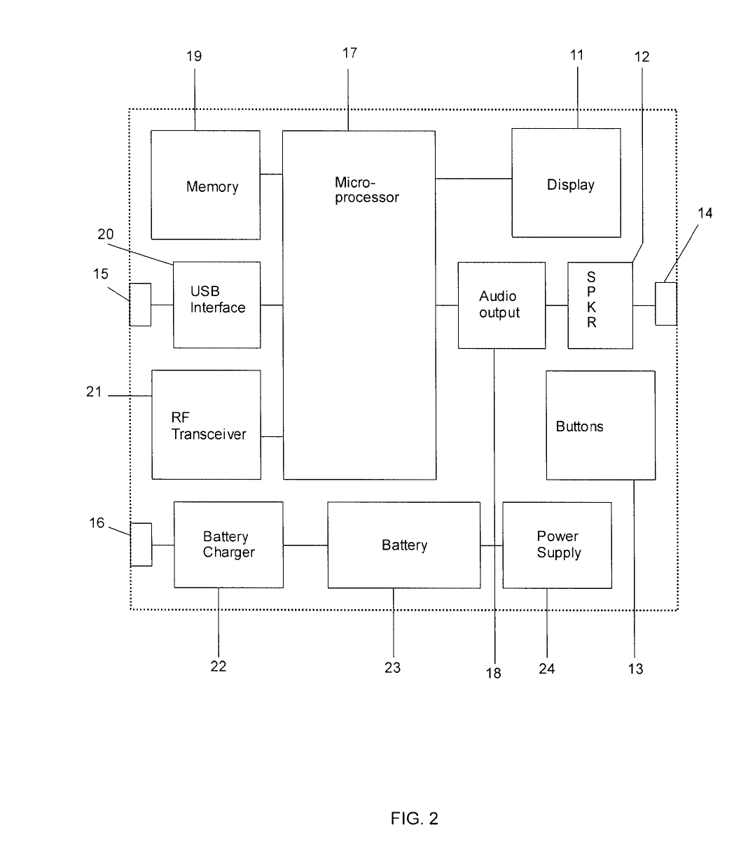

Referring to FIG. 2, a block diagram of the remote control 10, the invention can be explained. The remote control 10 comprises a microprocessor 17 that can have an interface that allows it to connect to: a large memory device 19, a USB interface chip 20, an RF transceiver 21, an audio generation circuit and amplifier 18, a speaker 12, a headphone jack 14, and USB jack 15. The remote control 10 allows user to input data via a variety of buttons 13 and view information from the remote control 10 through a display 11. Additionally, the remote control 10 will contain batteries 23, a power supply 24 to regulate power from the batteries and a charger circuit 22 that will allow recharging from an external power source via the external power jack 16. The microprocessor 17 can tolerate a wide variety of operating voltages such that it can run on variable battery power while all other circuitry within the remote control 10 may be powered down. When the microprocessor 17 senses user activity at the user interface keys 13, it will power up the required circuitry, such as the power supply 24, the display 11, audio output 18, etc. The batter life is maximized by powering only those components that perform currently active functions.

The remote control 10 can communicate with other system components through the RF transceiver 21. In different embodiments, the remote control 10 may use any of several standard commercially available RF transceiver 21 modules, allowing for a variety of modulation methods and transmission frequencies, enabling compatibility with multiple insulin pump systems. In an embodiment, the RF transceiver 21 modules may be interchangeable so that an RF transceiver 21 module that is compatible with the other system components can be easily attached to the remote control 10. In an embodiment, the remote control 10 can include different settings that allow the RF transceiver 21 to output RF signals that are compatible with the insulin pumps produced by various manufacturers.

The battery charger 22 is designed to accept input from an external power source as applied to the external power jack 16. As a convenience and safety feature for vision impaired users, the input voltage may be of any polarity and of a wide input range to allow use with any of a wide variety of external power sources. The battery charger 22 will also accept power input from the USB port 15 when that port is connected to a PC.

The battery 23 can be of a rechargeable type, minimizing the need for battery replacement by the user. The internal power supply 24 is a high efficiency switching type that maintains constant voltage output regardless of declining battery voltage. The power supply can be put into standby mode by the microprocessor 17 to minimize power drain when the remote control 10 is not being used.

### B. Audible Menus

In an embodiment, the remote control 10 implements an audible menu, such that speech is generated as a response to user operation of the buttons 13. The speech capability is particularly useful for blind users of the remote control 10. By providing audible signals indicating the current operation of the remote control 10, the buttons 13 may allow a blind user to operate the remote control 10 and accurately control the operation of the insulin pump 6. For example, an initial button press may bring the remote control 10 from a power down state to an on state, and the microprocessor 17 will access prerecorded speech from memory 19 and play that speech through the audio output circuit 18 to the speaker 12, or the headphone jack 14, and say the audible prompt, “Main Menu.” In another embodiment, the remote control 10 may internally synthesize the required speech as needed using a method called “text to speech” or TTS. The user, once having learned the operation of the remote control 10 may press the button corresponding to the “down” or “next” command, would then hear speech corresponding to the next menu item. By using the buttons 13, menu items can be selected and data can be input into the remote control 10.

The user, through appropriate button presses is able to listen and navigate all the available functions within the remote control 10. Once a desired menu item is reached, the user would then activate that function by pressing the “select” button. In the preferred embodiment of the invention the select button is located centrally to the other buttons. The four triangular shaped buttons as shown in FIG. 1 correspond to menu functionality corresponding to their physical placement; up, down, left and right. Depending on the particular function or menu operation being performed, various buttons may take on additional meanings. The left button, for example, could mean “go back”, or “escape”, or “cancel”. It should be noted the triangular shapes depicted in FIG. 1 are intended as examples. In other embodiments the buttons 13 may have shapes other than triangular.

Upon activation, by a user pressing an appropriate key 13, the remote control 10 normally begins operation at what is referred to as the “main menu”. The main menu is essentially a list of primary functions. The list of primary functions is shown on the LCD screen 11. The remote control 10 also speaks the name of the menu that has been activated, in this case “main menu” is spoken. The indicated functions may be chosen by the user pressing an up or down button (key).

The menu is a scrolling menu, such that if the bottom of the screen is reached and if there are more items in the menu list then the list moves up by one. The menu “revolves” so that when the bottom of the menu list is reached and the user presses the “down” button again, the remote control 10 will go back to the top of the menu list. Conversely, if the highlighted item in the menu list is the first on the list, and the user presses the up key, the menu list will now highlight the last item on the list. As each item is highlighted, the remote control 10 speaks the names of those functions. It should be noted that the spoken menus may not always be the same as the visible text for two reasons, 1) speech clarity and 2) screen text abbreviation (size limitation). The user, once having highlighted/listened to the desired function choice, can press the “select” button, thus activating the chosen function. The chosen function may be a sub-menu, in which there is now a list of new menu items to choose from. In this way, the remote control 10 is organized such that similar functions are grouped for convenient access. In an embodiment a sub-menu can lead to still more menus, until finally the desired function is reached.

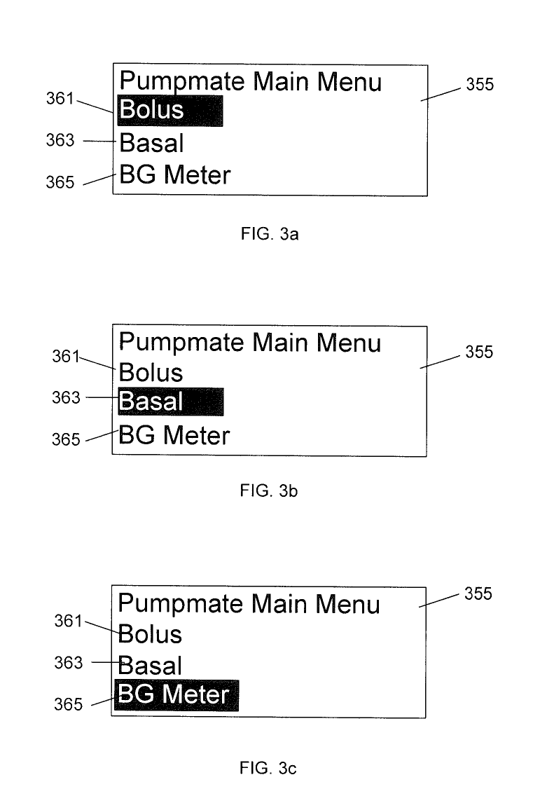

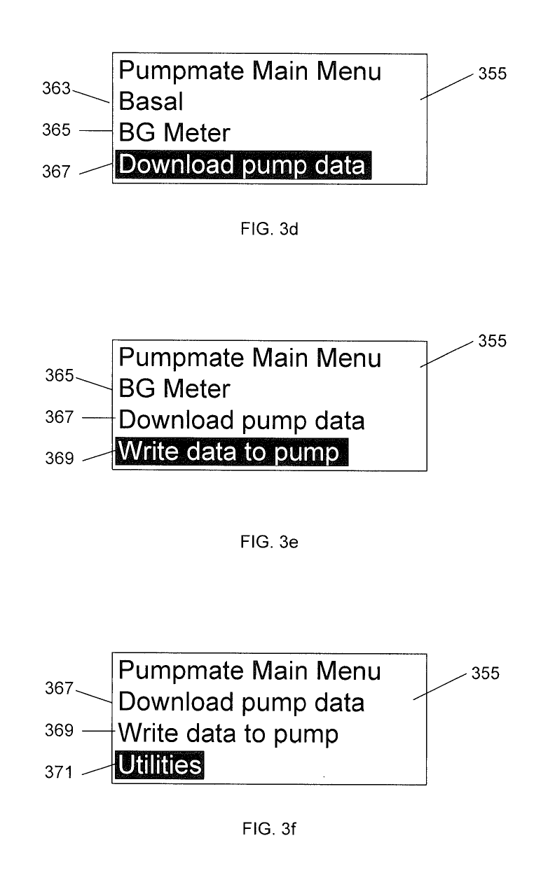

With reference to FIGS. 3a-3f, a series of remote control display screens 355 showing the main menu are illustrated. The remote control will indicate the choice of main menu item on the screen by displaying the text representing that function in a reverse video style, or highlighting 357. The remote control 10 may also speak the name of the chosen function, allowing a blind or sight impaired person to be aware of the menu position/highlighted item. When the user presses the up or down key to access the next or previous item, that item is then highlighted and spoken as well. The main menu includes the following listings: Bolus 361, Basal 363, BG Meter 365, Download Pump Data 367, Write Data to Pump 369, and Utilities 371. With reference to FIG. 3a, the display 355 has room for three menu items and Bolus 361 is highlighted. As discussed, the remote control may say “Bolus” when the word Bolus 361 is highlighted. The user can select Bolus 361 to use the bolus features or press a navigation button to go to a different function. If there is a period of inactivity, the remote control may repeat the word Bolus after a predetermined period of time.

With reference to FIG. 3b, when the user presses the down button, the highlighted text goes from Bolus to Basal and the remote control can say the word basal. Again, the user has the options of selecting the basal function or going to a different function. With reference to FIG. 3c, after the user has pressed the down button and the words BG Meter 365 are highlighted and the remote control can say “blood glucose meter.” With reference to FIG. 3d, if the user presses the down button again, the displayed functions will begin to scroll which adds words to and removes words from the display. The words, Download Pump Data 367 are highlighted and the word Bolus 361 is removed from the display 355. With reference to FIG. 3e, if the user presses the down button again, the remote control will highlight Write Data to Pump data 367 and say these words. The word Basal 363 can be removed from the display 355. With reference to FIG. 3f, if the down button pressed, the word, Utilities 371 is displayed and the remote control says utilities. Pressing the down button again will cause the main menu to scroll over to the first item again, Bolus 361.

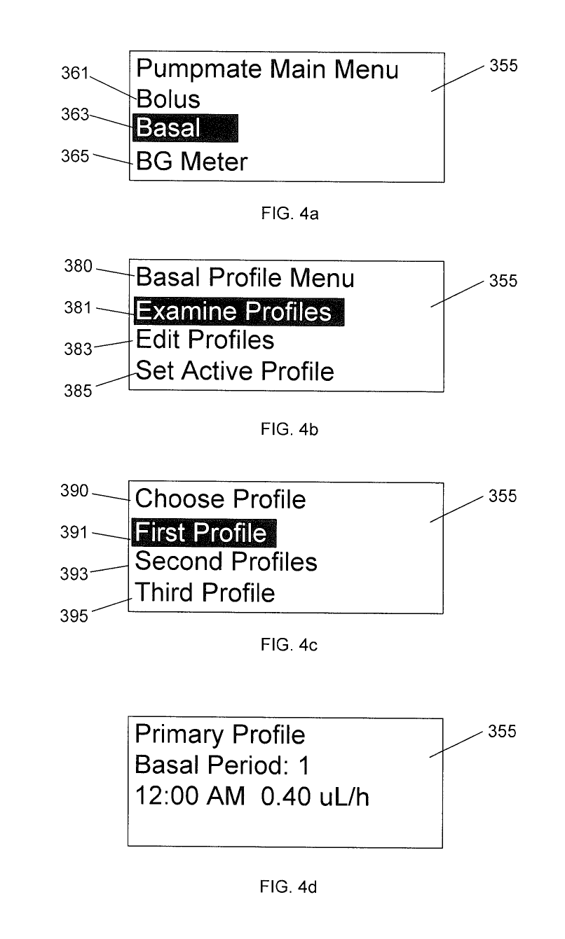

With reference to FIGS. 4a-4d, an example of a series of possible submenus is illustrated. In FIG. 4a, the main menu is illustrated and the word Basal 363 is highlighted. If the user presses the select button on the remote control, the display 355 will change and a first submenu can be displayed. With reference to FIG. 4b, the first line of the changed display 355 is “Basal profile Menu” indicating that the basal function has been selected. The remote control may say basal profile menu 380. The basal profile submenu 380 includes different menu items than the main menu. In this example, the basal profile submenu includes: Examine Profiles 381, Edit profiles 383 and Set Active Profile 385. With reference to FIG. 4c, if the user selects Examine Profiles 381, an examine profiles submenu 390 is displayed which can include the functions: Primary Profile 391, Second Profile 393 and Third Profile 395. With reference to FIG. 4d, if the Primary Profile 391 is selected, the Primary Profile is displayed. In this example, the Primary Profile indicates that the Basal Period 1 starts at 12:00 AM and is set for 0.40 uL/h.

The remote control 10 can also have additional buttons not shown in FIG. 1. In a preferred embodiment, an upper left button can function as a “power-on” button that also functions as a help button while the remote control 10 is powered on and operating. The remote control 10 can power down automatically after a predetermined period of non-use or timeout.

### C. Speech Tutorials

In an initial mode of operation, the remote control 10 can begin a tutorial session. The tutorial will describe the basic operation of the remote control 10 including: the menu system, sub-menus, help functions, control keys and activity monitoring functions that remind a user about what the user was doing if the remote control 10 detects that there has been a lapse in activity and cancels the function after there has been a long interval of inactivity.

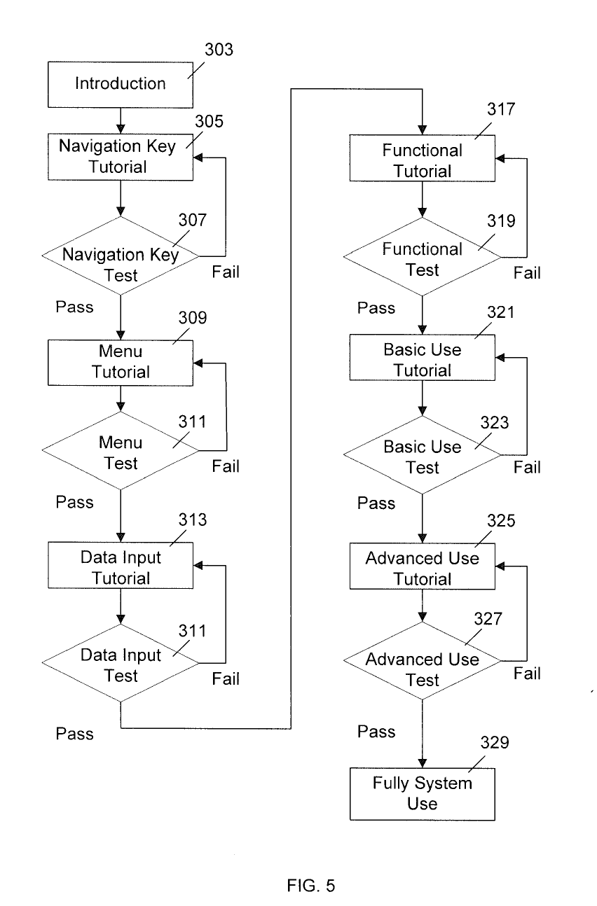

With reference to FIG. 5 a flow chart of the speech tutorial is illustrated. Before the inventive remote control is given to a new user, some basic training on the use of the remote control with the insulin pump and blood glucose meter may be necessary so the user understands the very basic operation of the system such as where is the on/off switch. The user can then begin to use the remote control which will have a variety of settings in memory that keep track of the user's activity. The remote control will be aware that this is the first time use by the user. The remote control will begin by offering to do an introduction tutorial 303, as if a sighted person was assisting and doing the tutorial. Initially, the system would talk about the remote control and describe the locations of the buttons and the basic functions 305.

The tutorial can begin with audio instructions emitted by the remote control. The following is an example of audio instructions that can be emitted by the remote control. “Please listen to a brief tutorial on the usage of the remote control and its functions. Once you learn the navigation button and menu functions you will be able to come back to any section of the tutorial for a refresher. First, let's learn what the push buttons on the front of the unit do. These may also be called keys, like the keys on your computer keyboard. There are seven keys, locate them now and press a few of them. I will read them back to you.”

In response to these instructions, the user presses one or more navigation keys, the pump reads back the descriptions of the keys that have been pressed, for example “left key”, “right key”, “select key”, etc. After several presses, or a short delay, the remote control can proceed to the next lesson. The remote control can then describe the functional keys, their location, their shape and the names of the keys and may also describe the functionality of the functional keys 305. After describing the keys, the remote control can test the user to try and press the right keys when prompted 307. The remote control will emit key descriptions and wait for a predetermined period of time for the user to press the correct key. The remote control will respond to the user's actions by emitting an audio signal that the correct key was pressed or that the incorrect key was pressed. The remote control may provide a hint and then give the user an additional opportunity to press the correct key. When a high accuracy control key rate is obtained by the user, the remote control can determine that the user understands the keys and has passed this portion of the training. The remote control can proceed with further instructions. If the user fails the test, the remote control can repeat the tutorial 305 before testing 307 the user again.

After the navigation test is passed, the remote control will talk about “speech menus”, what the speech menus mean, how they work in a menu tutorial 309. The user will be given a practice menu to try out. The remote control will emit a listing of menu items. The user will respond by navigating through the menus to select items in the test menus. The user will learn to use the ESCAPE key to go back to the main menu. The remote control will then explain sub-menus, which are simply more functions organized by logical groups, that make them easy to find. The system can test the user by asking the user to select an item in a sub-menu and wait for a predetermined period of time for the user to select the requested menu item 311. The remote control will respond to the user's actions by emitting an audio signal that the correct menu item was selected or that an incorrect item was selected. The remote control may provide a hint and then give the user an additional opportunity to select the correct menu item. When a high accuracy rate is obtained by the user, the remote control can determine that the user has passed the menu listings test and the remote control can proceed with further instructions. If the user fails the menu test, the menu tutorial 309 can be repeated.

The remote control will explain the various kinds of data that may be entered into the system in a data input tutorial 313. This could be a simple number, the time, a basal rate, serial number, etc. The first example will be entering a number. The number entry is done by telling the user to press the up or down keys which correspondingly increase or decrease the number. The value of the number will be spoken to the user with each change. The remote control will explain how to “select” a given value, or to ignore changes they may have made. The user will be given a quick skill check with an actual example. The remote control will give examples of the various number types and the user will try to input the requested data 315.

In an embodiment, the remote control can implement a rapid change function such that the user can press the up or down key multiple times while the pump is speaking. It is easy and intuitive to press the up or down key to change a value multiple times. The next value read out can be larger or smaller in amounts equal to the number of up/down key presses that occurred while the remote control was emitting the last value, allowing the user to quickly reach the desired value. In other embodiments, the value increments can change more rapidly when the control key is pressed and held. Again, the system will test the user to determine if the proper numbers or data can be input within a predetermined time period. Similarly, text input can be input by the keys as well. The system may read out the alphabet in sequence and the user may press a button to confirm a specific letter. If the text or word is identifiable based upon the input of less than all the letters, the system may transmit the expected word as an audio signal and then the user can press a confirmation button if the word is correct. Alternatively, the user may press a reject button if the expected word is incorrect. When the user has completed all of the control and data input training, the remote control begins describing the functionality as pertains to the insulin pump in a functional tutorial 317. Various examples of the remote control functions are described below. The remote control will provide instructions on how to access these functions through the keys and also provide instructions on how to obtain help if the user needs assistance. After the tutorial, the remote control can test the user on the functional features of the system 319. If the test is passed, the remote control can proceed to the next lesson and if the user fails, the system can repeat the functional tutorial 317.

After the functional test is passed, the remote control can instruct the user on the basic actual use of the system in a basic use tutorial 321. The remote control can then give the user a basic use test 323. If the user passes, the system may allow the user to operate some of the basic controls of the insulin pump. As the user becomes more proficient, the remote control will allow the user to control basic functions of the insulin pump. The system will provide checks that will ask the user to confirm controls.

After the basic use test is passed, the remote control 10 can describe the more advanced features of the system in an advanced use tutorial 325. The remote control can test the user in these advanced use features 327. If the user passes the test, the system can allow the user to full system use 329. If the advanced use test is failed, the remote control can repeat the advanced use tutorial 325.

It should be noted that while the majority of the discussion has centered around speech based menus, the same functions are also available by viewing the menu list and options on the included display. Characters shown are in large fonts, to assist persons with poor vision, who do not want to use speech. Additionally, the speech output option may be turned off. In an alternate embodiment of remote control 10, the display may be removed all together and the size and shape of remote control 10 will be reduced and altered.

### D. Remote Control Functions

The functions of the remote control can include: 1. general operation, 2. blood glucose meter management, 3. basal profiles, 4. boluses, 5. System limit settings, and 6. utility functions. These functions are described below. In other embodiments, various other functions can be performed by the remote control.

1. General Operation Of Remote Control

An insulin pump can contain many types of data and settings that will be subsequently explained such as basal profiles, basal limits, bolus limits, settings for glucose meters, settings for continuous blood glucose monitoring systems, settings for automatic bolus calculations, etc. In an embodiment the user will access the “read pump data” function via the menu system. The remote control 10 will initiate RF communications with the insulin pump 6 and in one operation will retrieve all the data and settings as mentioned above. The remote control 10 will confirm the completion of the operation visually on LCD screen 11 and with speech. The user may now access that information visually or audibly via the menu system. In a preferred embodiment the remote control 10 maintains a copy of all this data, allowing the user to view or edit this data without disturbing the operation of the insulin pump 6. In an embodiment the remote control 10 will make a working copy of this data locally within the remote control 10. The user may edit this data without risk of losing the unmodified copy also contained within the remote control 10. Once the desired changes have been made, the user can initiate RF communication with the insulin pump 6 and the changes will also be made on the pump. In the preferred embodiment the remote control 10 will then verify correctness of said changes, reporting the result visually on LCD screen 11 and with speech, if enabled, to the user. Once the data exchange is verified the remote control 10 will update its local copy of the insulin pump settings, ready for the next user access. For some individuals, especially those that are blind or sight impaired, learning and becoming proficient in the operation of an insulin pump can be confusing and intimidating, and a new user may make mistakes. In an embodiment the remote control 10 will maintain a copy, that is time and date stamped, of the very first data transfer with all settings and value from the insulin pump 6. This first set of data and settings may be restored to the pump by accessing a pump restore function from within the menu system, returning the insulin pump 6 to its original state. In an embodiment, the remote control 10 will maintain multiple time date stamped backup copies of pump data and settings such that as a user makes changes to the insulin pump settings and operation, the user may review the changes that were made or if desired return the insulin pump 6 to any one of those previous states.

2. Blood Glucose Meter Management Functions

Insulin is necessary for processing food that humans eat. A diabetic either does not produce any insulin or does not produce enough insulin. A diabetic must test his or her blood glucose level frequently to determine if they need additional insulin. A diabetic will utilize a blood glucose meter in combination with a “test strip”. Placing a drop of blood on the test strip will allow the blood glucose meter to determine the diabetics blood glucose level, typically measured in mg/dL (milligrams per deciliter). The diabetic can now determine what, if any, changes need to make to the pump settings that control insulin flow rate. These changes can be in the form of a bolus or a change to a basal profile. There are a plethora of blood glucose meters available, but they vary widely in usability for a vision impaired diabetic. Some only display the readings, some display and speak the readings, others display and transmit the reading directly to a compatible insulin pump, which may then assist the diabetic in determining adjustments to his insulin. The remote control 10 can facilitate the management of blood glucose data for sight impaired individuals by several means, such as receiving a blood glucose meter reading wirelessly, entering a blood glucose reading manually, reviewing past blood glucose readings, transmitting blood glucose readings to an insulin pump, and intercepting blood glucose readings from a blood glucose meter that would have been transmitted directly to an insulin pump.

2a. Receiving A Blood Glucose Reading From A Compatible Blood Glucose Meter

In an embodiment the blood glucose meter, remote control and insulin pump can communicate wirelessly. The remote control does not listen for blood glucose reading signals continuously, only when it is instructed to do so by the user. In order to actuate this function, the user would turn on the remote control and find the blood glucose meter menu from the main menu. The user can then choose the blood glucose sub-menu item that says: “receive blood glucose reading”. The remote control can then wait for a predetermined period of time (for example, up to 3 minutes) while the diabetic finds a “test strip”, inserts it into the end of the blood glucose meter, pricks his finger, and places a sample of blood on the end of the test strip. The blood glucose meter takes a few seconds to analyze the sample then displays the reading on its screen. Some meters transmit their readings via RF simultaneously if so equipped and enabled to do so.

The remote control will receive the RF transmission of blood glucose data, display them on LCD screen 11 and will emit the readings as audio signals for the user to hear, allowing the sight impaired to use this type of non-speaking blood glucose meter. The remote control will save the received blood glucose reading along with a time-date stamp. The user can also browse the blood glucose sub-menu for the most recently received blood glucose reading, to view or hear the reading another time. In an embodiment, multiple blood glucose readings are stored in a memory of the remote control with the time and date of the measurement, being made available for later access either through user menu control of the remote control 10, or USB access via external computer.

2b. Manually Entering A Blood Glucose Reading Into The Remote Control

The function of manually entering a blood glucose reading into the remote control is for those people that may have a blood glucose meter that doesn't transmit RF blood glucose signals. The user may determine the current blood glucose level from the meter and then enter the reading manually. After the blood glucose level has been entered, it can be treated the same as a reading that may have been received via RF transmission. It can be given a time-date stamp and stored for future access, and the user can actuate another menu item in the remote that causes the remote control to send the reading as an RF transmission to the insulin pump.

2c. Transmitting a Blood Glucose Value to an Insulin Pump

The remote control 10 user can access the blood glucose meter menu and navigate to the transmit blood glucose value function. The user is given a choice of escaping the function, or activating by pressing the select key. Upon activation, the remote control 10 emulates the RF transmission method of the target insulin pump and transmits the blood glucose data. In an embodiment the remote control 10 informs the user visually and through speech that the transmission was accepted by the insulin pump. In an embodiment, the remote control may, for reasons of safety, prevent the transmission of a blood glucose reading or manual entry if it is more than 15 minutes old.

2d. Intercepting a Blood Glucose Meter RF Transmission

Typically, an insulin pump is configured to automatically receive readings transmitted by a compatible blood glucose meter. There are many reasons blood glucose reading can be incorrect, such as a defective test strip, or insufficient blood being applied to the test strip, so the user may wish to check the blood glucose readings before they are automatically sent to the pump. In some cases a user may want to take a second reading, or even a third and decide which is correct and transmit only the correct reading to the insulin pump using the remote control 10. In an embodiment where the remote control 10, blood glucose meter 1 and insulin pump 6 communicate with RF signals, and in which device serial numbers are used to identify RF transmissions from said devices, the remote control 10 can be used to effectively intercept blood glucose reading prior to their receipt by the insulin pump 6. When the intercept function of the remote control 10 is enabled, through access by the user with the appropriate menu function, the remote control 10 will initiate communication with the insulin pump 6 and authorize the pump to receive transmissions from the remote control 10 as though it were the blood glucose meter, and the remote control 10 can remove the serial number assignment that exists within the insulin pump 6 that allows it to receive transmissions from the blood glucose meter. The remote control 10 can now receive RF transmissions from the blood glucose meter 1, display and annunciate those readings with speech, thus, allowing the user to decide if the reading is valid. If the user agrees the reading is valid, the remote control 10 will now transmit those readings to the insulin pump, as described above.

3. Basal Profiles

In an embodiment, the remote control can be used to control or modify a pattern of delivering basal insulin to suit the needs of the user. For example, the remote control can reduce a basal at night to prevent low blood sugar in infants and toddlers, increase a basal at night to counteract high blood sugar levels due to growth hormone in teenagers, increase a basal at pre-dawn to prevent high blood sugar due to the “dawn effect” in adults and teens. Various other pattern modifications are available. In other embodiments, the remote control can include a proactive basal plan before regularly scheduled exercise times such as morning gym for elementary school children or after school basketball practice for high school children.

Basal insulin requirements will vary between individuals and periods of the day. In an embodiment, the remote control can be used to determine the basal rate. The basal rate for a particular time period is determined by fasting while periodically evaluating the blood sugar level. Neither food nor bolus insulin must be taken for 4 hours prior to or during the evaluation period. If the blood sugar level changes dramatically during evaluation, then the basal rate can be adjusted to increase or decrease insulin delivery to keep the blood sugar level approximately steady. For example, to determine an individual's morning basal requirement, the user must skip breakfast. On waking, they would test their blood glucose level periodically until lunch. Changes in blood glucose level are compensated for with adjustments in the morning basal rate. The process can be repeated over several days, varying the fasting period, until a 24-hour basal profile has been built up which keeps fasting blood sugar levels relatively steady. Once the basal rate is matched to the fasting basal insulin need, the pump user will then gain the flexibility to skip or postpone meals such as sleeping late on the weekends or working overtime on a weekday.

Many factors can change insulin requirements for a user and require an adjustment to the basal rate. These factors include: continued beta cell death following diagnosis of type 1 diabetes, growth spurts particularly during puberty, weight gain or loss, any drug treatment that affects insulin sensitivity (e.g. corticosteroids), eating, sleeping, or exercise routine changes, whenever the control over hyperglycemia is degrading, adjustments based upon changes in the seasons and other factors. Since the basal insulin is provided as a rapid-acting insulin, the basal insulin can also be immediately increased or decreased as needed with a temporary basal rate.

In yet another embodiment, the remote control can be configured to treat a low blood sugar level or a low glycemic food with a bolus after a meal is begun. The blood sugar level, the type of food eaten, and a person's individual response to food and insulin have an impact on the ideal time to bolus with the pump. These preferred times can be determined and programmed or directly input into the remote control.

3a. Create/Edit Insulin Pump Basal Rate Profile

A basal rate simply stated is a specific rate of insulin flow, typically measured in ul/HR (microliters per hour) for a specific time period. A basal profile is a collection of one or more time periods, each with a specific basal flow rate, concatenated together. Further, a basal profile typically covers a 24 hour period starting at 12:00 AM. A basal profile reflects the needs of one particular diabetic for that 24 hour time period. Depending on the individual, multiple basal profiles, and the ability to choose which profile is being used is desirable. A diabetic may have a profile designed for days of physical exercise, or perhaps another for a regular work day, or another still for a day off, or yet another if the diabetic is temporarily ill. Because so many things affect the needs of a diabetic, the ability to quickly edit and/or create a profile are important. The insulin pump may allow for 3 basal profiles, and the remote control 10 may also allow a like number of basal profiles to be managed. In an embodiment the remote control 10 can also provide for a larger number of basal profiles to be maintained, and when needed, a particular basal profile may be transmitted to the insulin pump 6 in place of one of the limited number of profiles it is capable of managing. As described above, a basal profile is basically a set of start times and insulin flow rates. For example with reference to FIG. 5, a basal profile may look like: (12:00 am-1.0 ul/Hr), (9:00 am-1.5 ul/Hr), (9:00 pm-0.75 ul/Hr). This would be interpreted as a basal profile with 3 time periods, starting at 12 AM, 9 AM and 9 PM, each time period will have a different insulin flow rate.

In order to access the basal rate function, the user will navigate to the basal menu. The user can choose from reading a profile, editing a profile, and other functions. If the user chooses EDIT, then the user “sees” another menu where the user will choose which profile to edit. Upon selecting a profile, the user is then presented with the time of the first time slot, or time period. The first time slot or time period may start at 12:00 AM. The remote control can read the value by emitting basal rate values such as, “zero point two five” (0.25). The user will then press the up or down key to change the basal rate value. If the user presses the up button, the remote will now emit the next higher basal rate value, for example, “zero point three zero” (0.30), and so on. If the user has a long way to go to get to the desired reading the user can press the up key (or down) multiple times during the interval in which the remote control is audibly emitting the current reading. For example, if a user were to press the up key 5 times during the audio output, the next reading from the remote control may be “zero point three five”. In this manner, the user can more quickly get to the desired flow rate reading. Basal rates can vary widely however, from user to user. An insulin pump may allow a maximum basal rate of 35.00 ul/H (microliters per hour). In an embodiment, the remote control can be programmed to prevent the flow rate from exceeding this value or a smaller value based upon a doctor's requirement.

It can take an extremely long time to change the flow rate value by increments of 0.05 units at a time. In an embodiment, the remote control allows the user to press the left arrow key to change the units of increment. In this case, the remote control will change units by plus or minus 1.00. For example, if the desired flow rate is 15.75 ul/H, the user can switch the increment value to change by ones using the left key and get to 15.00 very quickly, then by pressing the right key, the units of change will now drop back down to 0.05 or other similar small increment and the user can fine tune the value. Once the first time period is set, the user will press SELECT, or if in the 0.05 change rate, the user can press the right key too, the user will be given the opportunity to edit/create the next time period value. If the user is editing an existing profile and the next time period of the basal profile already exists, the user will be audibly informed, and given a choice of continuing to use the settings for that time period, or the user can end (truncate) the profile with the period just edited/created. This can be done by pressing the right key to continue and the down key to end.

In the second or other subsequent basal time period, the user can edit the start time of each period as well as the basal rate. The editing is done the same way. The up key increases the second period start time and the down key reduces it. Again, the user can press the up or down keys multiple times while the remote control is outputting the current value to allow the user to reach the desired value quickly. The right key takes him to the basal edit with the 0.05 rate set. The left key takes him to basal edit but with a change value of 1.0. A left key again puts him back into time edit. Once a basal profile edit has been completed the remote control outputs the settings and user listens to a summary of the changes. This insures that the right values have been set since wrong basal values can result in injury to the user. Once the review is complete, the user can choose to ignore the changes, or accept them (escape or select).

At any point during an edit sequence the user can press ESCAPE which will stop the basal profile editing function. The remote control can have various help level settings and when the help level is set to “medium”, the user will be asked to confirm the “escape”. At any time during this menu, the user can also press the “power/help” button and be given guidance.

3b. Transmit Basal Rate Profile To Insulin Pump

In an embodiment, the remote control can subsequently transmit any basal profile changes to the insulin pump. The user can navigate to the “transmit to pump” function of the remote control 10 and initiate transmission. The remote control will only transmit to the insulin pump if some change to a basal profile, or other setting has been made. The remote control can then verify that the basal settings were properly sent and reports success or failure of the setting changes to the user.

The basal profile data are stored in non-volatile memory, such that memory is retained without power. The batteries can be removed from the remote control and the basal profile settings or any other settings will not be lost. In an embodiment the remote control 10 keeps note of what changes a user has made so that RF transmissions to the pump and changes to the pump are minimized.

4. Boluses

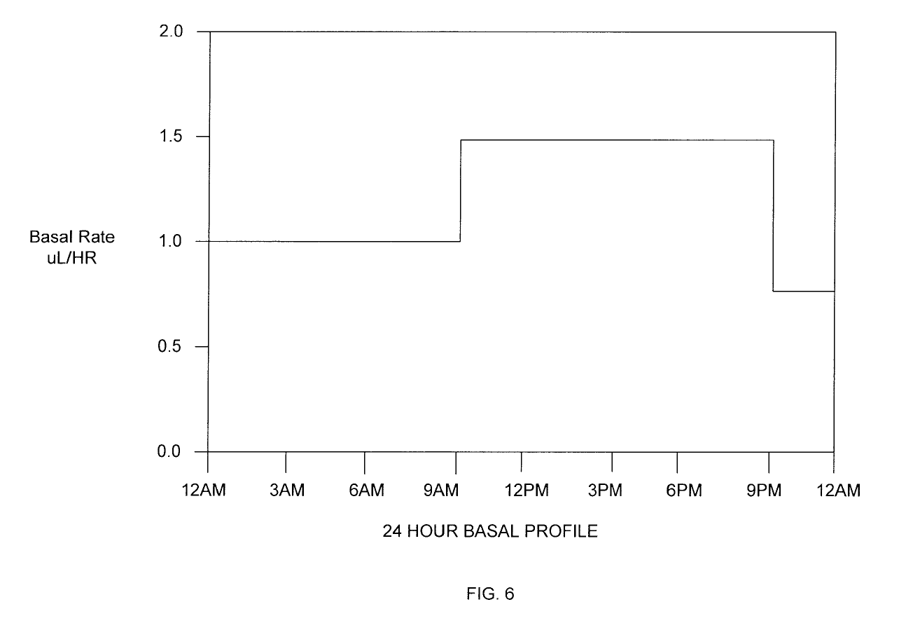

The insulin pump delivers a single type of fast-acting insulin in two ways: 1) a bolus dose that is pumped to cover food eaten or to correct a high blood glucose level and 2) a basal dose that is pumped continuously at an adjustable basal rate to deliver insulin needed between meals and at night. An insulin pump user has the ability to influence the profile of the rapid-acting insulin by shaping the bolus. While each user must experiment with bolus shapes to determine what is best for any given food, they can improve control of blood sugar by adapting the bolus shape to their needs. FIGS. 6-9 illustrate four graphs of different example bolus shapes that illustrate the units of insulin provided over a period of time.

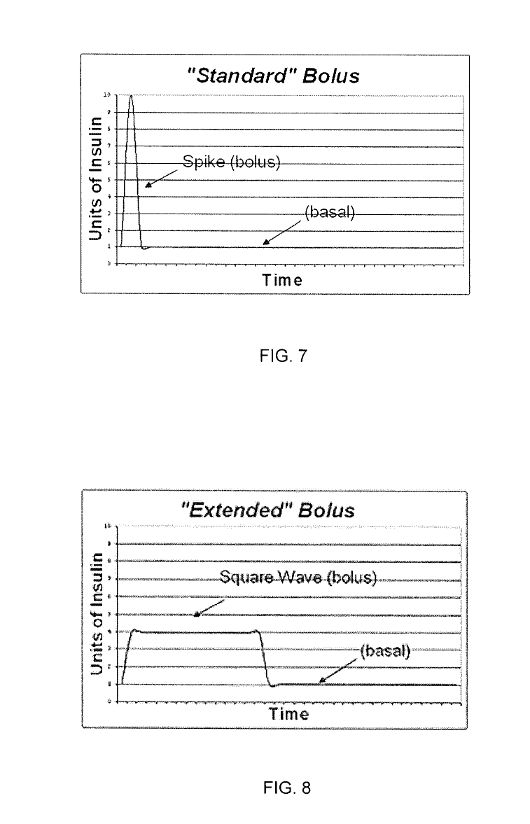

With reference to FIG. 7, a graph of a standard bolus is illustrated. The standard bolus is an infusion of insulin pumped completely at the onset of the bolus. It is most similar to an injection. By pumping a large volume of insulin with a “spike” shape, the expected action is the fastest possible bolus for that type of insulin. The standard bolus can be applied when eating high carbohydrate, low protein, and low fat meals because the standard bolus will return blood sugar to normal levels quickly. After the bolus is complete, the insulin pump will return to its normal basal insulin flow rate.

With reference to FIG. 8 a graph of an extended bolus is illustrated. The extended bolus is a slower infusion of insulin spread out over longer period of time than the normal bolus. By pumping with a “square wave” shape, the bolus avoids a high initial dose of insulin that may enter the blood stream and cause low blood sugar before digestion can facilitate sugar entering the blood. The extended bolus is appropriate when a user eats a high fat, high protein meal such as steak, which will be raising blood sugar for many hours past the onset of the bolus. The extended bolus is also useful for those with slow digestion which can be caused by gastroparesis Coeliac disease or other conditions.

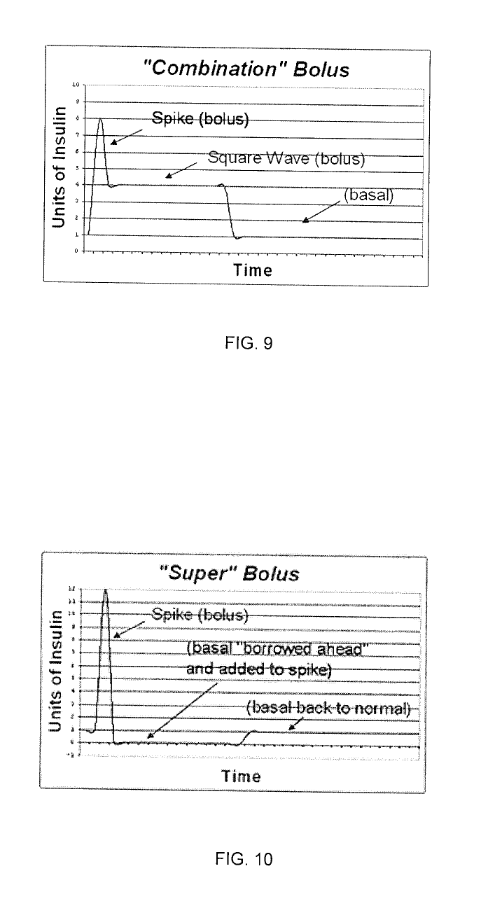

With reference to FIG. 9, a graph of a combination bolus is illustrated. The combination bolus combines a standard bolus spike with an extended bolus square wave. This combination bolus shape provides a large dose of insulin initially, and then also extends the duration of the insulin flow rate. The combination bolus is appropriate for high carbohydrate, high fat meals such as pizza, pasta with heavy cream sauce, and chocolate cake.

With reference to FIG. 10, a graph of a super bolus is illustrated. The super bolus is a method of increasing the insulin flow rate above that of the standard bolus. Since the action of the bolus insulin in the blood stream will extend for several hours, the basal insulin could be stopped or reduced during this time. This facilitates the “borrowing” of the basal insulin and including the basal insulin in the bolus spike to deliver the same total insulin with faster action than can be achieved with a spike and basal rate together. The super bolus is useful for certain foods such as sugary breakfast cereals which cause a large post-prandial peak of blood sugar. The super bolus can attack the blood sugar peak with the fast delivery of insulin that can be practically achieved by pumping.

In an embodiment, the remote control can also be programmed to perform a pre-bolus. The pre-bolus improves upon the insulin pump's capability to prevent post-prandial hyperglycemia. A pre-bolus is simply a bolus of insulin given before the insulin is actually needed to cover carbohydrates eaten. There are two situations where a pre-bolus is helpful. In a first situation, a pre-bolus of insulin will mitigate a spike in blood sugar that results from eating high glycemic foods. Infused insulin analogs such as NovoLog and Apidra typically begin to impact blood sugar levels 15 or 20 minutes after infusion. As a result, easily digested sugars often hit the bloodstream much faster than infused insulin intended to cover them, and the blood sugar level spikes upward as a result. If the bolus were to be infused 20 minutes before eating, then the pre-bolused insulin will be hitting the bloodstream simultaneously with the digested sugars to control the magnitude of the spike. In a second situation, a pre-bolus of insulin can also combine a meal bolus and a correction bolus when the blood sugar is above the target range before a meal. The timing of the bolus is a controllable variable to bring down the blood sugar level before eating again causes it to increase.

4a. Issuing a Bolus

A user of the remote control 10 can issue a bolus by navigating to the desired function by means of the previously described menu system. In an embodiment, and referring to FIGS. 4 through 7, and by pressing the appropriate keys 13 the user can select the type of bolus that the user desires, followed by the size of bolus (units of insulin) desired, and finally the time duration (if needed). Emitting audible feedback, the remote control 10 allows confirmation of the chosen settings. The user can press the select key to begin the bolus, or alternatively the escape key to exit the function. Once selected, the remote control 10 can communicate with the insulin pump 6, causing it to begin a bolus operation with the desired parameters. The numeric entry of bolus size and time duration allow for multiple key presses while audible data is emitted, allow the user to rapidly reach the target values.

5. System Limit Settings

In an embodiment, the remote control can have features that limit the operation of the insulin pump. As a means of limiting mistakes by the user, especially first time users, one's doctor or trainer will most probably have set up the pump with various preset insulin limits, such as maximum bolus, maximum basal rate, and an initial basal profile. The user can also change these values at the pump or through the remote control. A user can access the bolus menu by pressing the up/down keys until you see “Bolus” menu is selected. The remote control can output a new menu, that may include the sub-menu items “Issue Bolus”, “Set Bolus Increment”, and “Set Max Bolus”. If the user wishes to set the maximum Bolus amount, this listing is selected and the remote control can display “Set Max Bolus Amount” and may speak the current max bolus amount setting, which is in units (1 u/L, microliter) of insulin. Using the up/down keys the user can change the setting. In this example, the maximum bolus that the pump allows is 25 units, so the remote control can never set the limit to more than the pump setting. However, the remote control can be set for limit that is less than the pump limit, for example, 10 or 12 units. The maximum basal rate and an initial basal profile can also be set in a similar manner.

6. Utility Functions

The utility functions are another set of sub menu-items that may be located in a “utilities” menu and can include but is not limited to: help level, tutorial menu, date and time, language, volume and display, reminder timers and insulin pump communication check.

6a. Help Level

The utility menu can include: “help level” as one of the menu items. If a user selects the help level utility function, the help level can be set by the user. The help level can depend upon the pump use skill level of the user with a high level of help needed for a novice while an expert may only require a low level of help or no help at all. The help levels can be for a numerical value 1, 2, or 3, or a text description, off, low, medium, high. The help level will change the amount of instruction speech provided by the remote control upon entering a given menu. For example, if the user is about to edit a basal profile with the help set to “high”, the remote control will explain the parts of the profile [(time, rate), (time 2, rate 2), etc.] and how you will edit them, including the use of the various keys. If the help level were set to medium, then the instruction text will be much more limited, saying things such as, “Editing profile number one. First set the start time, then the basal rate to use. Repeat this for each time period.” With the help level to set “off”, the remote control may only provide a very brief prompting and will be sent, such as; “editing primary profile”, and nothing else. The help level will also alter the information provided when the user presses the “help” key. When a user presses the help key, the remote control will re-play the help messages for whatever the help level is set at. If additional information is needed, the user can press the help key again and the remote control may switch the help level up and then provide information at the next higher level. This increase in help level can be a temporary one-time increase and the remote control may return to the normal help level setting after a help message has been transmitted. The remote control can also have a mechanism for stopping the help instruction if the user requests an excessively lengthy instruction or if the instructions were accidentally accessed by mistakenly pressing the help button. In an embodiment, the user can stop the help message by pressing the escape key.

6b. Date and Time Set

In an embodiment, the remote control may include a radio clock or radio-controlled clock that is synchronized by a date and time code bit stream transmitted by a radio transmitter connected to a time standard such as an atomic clock. Such a clock may be synchronized to the time sent by a single transmitter, such as many national or regional time transmitters, or may use multiple transmitters, like the Global Positioning System. In other embodiments, the clock may have to be set manually. The user can access the date and clock setting functions through the utility menu and set the time and date as described above. The date and time can be very important to the accurate processing of the basal rate profile. In an embodiment, the remote control 10 can set the time and date setting held within an insulin pump 6 to be the same as the remote control 10. In an embodiment, the user can choose to use the time and date settings currently held by the pump.

6c. Speech/Language

The remote control can be made to display text on the LCD screen 11 and to speak in various different languages. The user can select the language settings through the utility menu. Because the speech menus may not be recognizable, the language menu can be displayed on the display of the remote control and may initially be set by sight.

6d. Volume

The user can adjust the volume settings through the utility menu. In an embodiment, it may be desirable to have special volume settings based upon the use of headphone or RF ear piece. The remote control may also have a mute setting so that the sound does not disturb others in areas that require silence such as the symphony, opera, movie theaters, etc. In an embodiment the user may access the speech volume settings by a “hot key” selection, such as the simultaneous pressing of two keys.

6e. Access Tutorial Session and Knowledge Base

The user can access the described tutorial sessions through the utility menu. Each session may have a specific sub-menu identification. In an embodiment, the remote control can also have a knowledge base, which allows users to share information with each other. The knowledge base information can be downloaded from a website. The different subjects of the knowledge base can have different sub-menu listings.

In still other modes of operation, and without going into excessive detail, the remote control 10 may be connected to a PC via a USB connection where software updates may be made, log file information may be downloaded, language selection, voice options, may be downloaded. Easy software updates allow the remote control 10 to be updated with new firmware and features as customers require.

6f. Reminder Timers

A diabetic's life unfortunately is one of constant vigilance in regards to blood glucose measurements and frequent administering of boluses and adjustments to basal profiles. It is easy for anyone to lose track of time. In an embodiment, the remote control 10 can include several timer functions, accessible and settable through the menu system. A timer may be set to remind a user to measure his blood glucose level 30 minutes before meal time, or at any of several other times during the day. The remote control 10 can respond with a variety of emitted messages or tones to alert the user. Reminder times can also be used simply as a morning wake up alarm. In an embodiment a timer may be set to alert the user on a specific date and time.

6g. Insulin Pump Performance Check

An insulin pump 6 may exhaust its supply of insulin, or a low battery condition may exist. Typically an insulin pump will emit an audible alarm or vibration, but an individual who is a sound sleeper may not notice, or the insulin pump may be located under blankets or other material that may mask sound and vibrations. In an embodiment, the remote control 10 can be set to initiate communications with the insulin pump 6 to verify pump activity at periodic intervals, such as when the user may be sleeping and upon noting an insulin pump alarm condition, the remote control 10 can emit an audible speech alarm of ever increasing volume. Alternately, the remote control 10 may emit a distinctive alarm tone to alert the user to the pump condition. If located in a convenient place, such as the user's night stand or table, the audible alarm or message emitted can be easily heard.

While the invention has been described herein with reference to certain preferred embodiments, these embodiments have been presented by way of example only, and not to limit the scope of the invention. Accordingly, the scope of the invention should be defined only in accordance with the claims that follow.

1. A method for using an insulin pump comprising:
providing a remote control for the insulin pump having a controller, a speaker, control keys and an RF transceiver;
teaching a patient the locations of the control keys by the controller that includes emitting an audio identification of the control key that has been pressed by the speaker after each of the control keys is pressed;
providing a key location test by the controller that includes emitting a key identification request and an audio verification when the key corresponding to the key identification was pressed within a predetermined response time;
actuating the transmitter into an operating mode after determining that the patient can proficiently operate the remote control; and
transmitting control signals from the transmitter of the remote control to the insulin pump in response to the pressing of the control keys after determining that the patient can proficiently operate the remote control.

2. The method of claim 1 further comprising:
teaching a patient how to use audio menus by the controller that includes emitting the audio menu and emitting the menu item that was selected when the control keys is pressed;
providing an audio menu test by the controller that includes emitting a menu item request and one of the audio menus;
providing an audio menu verification when the control key corresponding to the menu item is pressed within the predetermined response time.

3. The method of claim 2 further comprising:
teaching a patient how to use audio sub menus by the controller that includes emitting the audio sub menu and emitting the sub menu item that was selected when the control keys are pressed;
providing an audio sub menu test by the controller that includes emitting a sub menu item request and one of the audio sub menus; and
providing an audio sub menu verification when the control key corresponding to the sub menu item is pressed within the predetermined response time.

4. The method of claim 3 further comprising:
teaching a patient how to get to a main menu by the controller that includes emitting the audio menu and emitting the main menu when the escape key is pressed;
providing an audio main menu test by the controller that includes emitting the main menu and a sub menu;
transmitting an audio request to return to the main menu; and
providing an audio main menu verification when the escape key is pressed within the predetermined response time.

5. The method of claim 1 further comprising:
teaching a patient how to enter data by the controller that includes emitting audio instructions for entering data and emitting the data input after a set of control keys are pressed;
providing a data input test by the controller that includes emitting a data input request, and an audio verification if the control keys corresponding to the data input request are pressed.

6. The method of claim 1 further comprising:
teaching a patient how to enter a number by the controller that includes emitting audio instructions for entering the number and emitting the number input after the control keys for changing the number are pressed and emitting a number verification after the control keys for selecting the number are pressed;
providing a data input test by the controller that includes emitting a data input request, and an audio verification if the control keys corresponding to the data input request are pressed.

7. A method for using an insulin system comprising:
providing a remote control having a controller, a speaker, control keys and an RF transceiver;
inputting a sequence of the control keys pressed in response to a set of menus that corresponds to a system command;
identifying the system command by the controller;
emitting an audio signal that corresponds to the system command;
transmitting the system command from the RF transceiver to a system component.

8. The method of claim 7 further comprising:
receiving a first blood glucose reading by the RF transceiver of the remote control;
wherein the system command is a blood glucose reading request transmitted from the RF transceiver to a blood glucose meter.

9. The method of claim 8 further comprising:
storing the blood glucose reading with a time and a date of the blood glucose reading in a memory of the remote control.

10. The method of claim 8 further comprising:
inputting a second blood glucose reading to the remote control; and
transmitting the second blood glucose reading from the remote control to an insulin pump.

11. The method of claim 10 further comprising:
inputting a command confirmation through the control keys before transmitting the system command to the system component.

12. The method of claim 7 further comprising:
starting a multi level timer after each of the control keys is pressed; and
emitting an audio reminder when the control keys corresponding to the first command are not pressed after a predetermined time period has elapsed from starting of the multi level timer; and
emitting an audio signal by the controller identifying the control keys pressed in the first sequence.

13. The method of claim 7 further comprising:
storing a maximum insulin pump flow rate in the remote control;
detecting by the controller that the system command is requesting more than the maximum insulin pump flow rate; and
adjusting the system command by the controller to the maximum insulin pump flow rate.

14. The method of claim 12, wherein the system command is for a bolus.

15. The method of claim 12, wherein the system command is for a basal rate.

16. The method of claim 12 further comprising:
emitting an audio signal from the speaker indicating that the system command has been adjusted to the maximum insulin flow rate.

17. The method of claim 7 further comprising:
storing the system command in a memory of the remote control;
wherein the system command is a first basal rate profile that includes one or more time periods, each of the time periods having an insulin flow rate and the insulin flow rates for the time periods are different.

18. The method of claim 16 further comprising:
transmitting the first basal rate profile system command as RF signals from the RF transceiver.

19. The method of claim 7 further comprising:
inputting a help level setting;
wherein the audio signal that corresponds to the system command includes information about the system command that is proportional to the help level setting with a low help level setting causing the audio signal to have less of the information and a high help level setting causing the audio signal to have more of the information.

20. The method of claim 18 further comprising:
inputting an escape key; and
stopping the inputting the sequence of the control keys.

21. The method of claim 7 wherein the system command is a bolus that includes an insulin volume.

22. The method of claim 20 further comprising:
inputting an issue bolus system command to the remote control; and
transmitting the issue bolus system command as an RF signal from the RF transceiver.

23. (canceled)

23. The method of claim 7 further comprising:
providing a data interface that is coupled to the remote control;
receiving software updates through the data interface; and
storing the software updates in a memory of the remote control.

24. The method of claim 23 wherein the data interface is a universal serial bus (USB) connection.

25. A method for using an insulin pump system comprising:
providing a remote control having a controller, a speaker, control keys and a transceiver;
receiving an RF signal by the transceiver from an insulin pump indicating a needed service;
emitting a signal from the remote control that corresponds to the needed service.

26. The method of claim 25 wherein the needed service is refilling an insulin tank on the insulin pump.

27. (canceled)

27. The method of claim 25, wherein the signal emitted by the remote control is an audio signal.

28. The method of claim 25, wherein the signal emitted by the remote control is a vibration signal.

29. The method of claim 25, wherein the needed service is replacing or recharging a battery on the insulin pump.

30. The method of claim 7 wherein the emitting the audio signal that corresponds to the system command is through a headphone jack of the remote control.
